SEQUENCE LISTING (6 sequences)

SEQ ID NO: 1 - DNA, 413 bp, Homo sapiens
gcatcaaccgcatggtgctgtgctacctcatccgcttcctgcaggtcttcgtgcagccggccaacgtcgcggtcaccaagatggatgtcagcaacctggccatggtgatggcgcccaactgcttgcgctgccagtccgacgacccgcgcgtcatcttcgagaacacccgcaaggagatgtccttcctgcgggtgctcatccagcacctggacaccagcttcatggagggtgtgctgtagcgggggcgcccggggacaggagggatgtcctgccgcccccagccaggccgaactccgcactcgctctcccggcagaggggccagaatcgcccggcccagccctggagccccctccactcccccaggcccctggccccggcgctccccacgtcttctgcctggtctgagggtgca

SEQ ID NO: 2 - DNA, 225 bp, Homo sapiens
atggtgctgtgctacctcatccgcttcctgcaggtcttcgtgcagccggccaacgtcgcggtcaccaagatggatgtcagcaacctggccatggtgatggcgcccaactgcttgcgctgccagtccgacgacccgcgcgtcatcttcgagaacacccgcaaggagatgtccttcctgcgggtgctcatccagcacctggacaccagcttcatggagggtgtgctg

SEQ ID NO: 3 - DNA, 1782 bp, Homo sapiens
ccgatgccctgaagctgcaggtggaccagtggaaggtgcccacaggcctggaagacccccacgtccctgcgtccctgctgaagctgtggtaccgggagctggaggagcccctgatcccgcacgagttctacgagcagtgcatcgcgcactacgacagccccgaggcggcggtggccgtggtgcacgcgctgccccgcatcaaccgcatggtgctgtgctacctcatccgcttcctgcaggtacttccctccccgggggtccccgctgctttcccctccccgccccgcccccgcccctccgcgcccaccctcgctcctccaccaggtcttcgtgcagccggccaacgtcgcggtcaccaagatggatgtcagcaacctggccatggtgatggcgcccaactgcttgcgctgccagtccgacgacccgcgcgtcatcttcgagaacacccgcaaggagatgtccttcctgcgggtgctcatccagcacctggacaccagcttcatggagggtgtgctgtagcgggggcgcccggggacaggagggatgtcctgccgcccccagccaggccgaactccgcactcgctctcccggcagaggggccagaatcgcccggcccagccctggagccccctccactcccccaggcccctggccccggcgctccccacgtcttctgcctggtctgagggtgcagccagggcacagcagcggcggggagggcgcctctggccccccacctcacggccagttcccgcgggcaccgcctcgccctccgctggccgcgggtcagctccgagaaagtgccttctgtgtcctggagccgagcgacgctgcctccttggggccgggctgcctccctgtggctcctgcgcgccctggcctgggccttgcccagccgccccggtctctccttccctttctcctgtcctcgtcctggcctgcagctcttcccagccccgagagagcttcccgacctgtccccgcctcctctccctccctcggcccgtggtccccagctggtgactgctcaggagtttgggggctccaggacagtgggcccggggcctggcaggctctcggtgggtggggtgggggcccccaaaccaaagtcctctggggtagggagcagggctgggcaggcattctgggggcagggtgggggaggggcgagagtatttttttcttcgtgtaactgtaaatccagaatctatcctgcatcgcagcccaccgtgtatagagatataaatagagggaaagatataagaactaaatttgctaatgacatagttttaacctaaatgctatttatctctgagccgtccccgtcctccgtgcagagcaagttgaggtcattccttcttttcttctccgatcttttttcttggcttctgaccaaaaaccaagctctaccccatccccatcccagacctgcaggagacgagcgagcgggaaggcgccgggcccgggactgtccgttctcggggccagagctgctgggggaccgagtttgtacattttccattttggaattttgagttccaattgttgtaaaacttaatttctccccagtttttatatatatattttttagagttccgtttttatttattaaaaacaaaagccccagccctgccgaggcctgggcggcgtcctcagtcgggtggtcccggggcctttgcggtcccgcccggctgagacgctcgccccgacgcatggacccgagaggcgacgacacgagtgaataaagtgcacatggaaaaaaaaaaaaaaaaaaaaaaaaaa

SEQ ID NO: 4 - DNA, 477 bp, Homo sapiens
atggtgctgtgctacctcatccgcttcctgcaggtacttccctccccgggggtccccgctgctttcccctccccgccccgcccccgcccctccgcgcccaccctcgctcctccaccaggtcttcgtgcagccggccaacgtcgcggtcaccaagatggatgtcagcaacctggccatggtgatggcgcccaactgcttgcgctgccagtccgacgacccgcgcgtcatcttcgagaacacccgcaaggagatgtccttcctgcgggtgctcatccagcacctggacaccagcttcatggagggtgtgctgtagcgggggcgcccggggacaggagggatgtcctgccgcccccagccaggccgaactccgcactcgctctcccggcagaggggccagaatcgcccggcccagccctggagccccctccactcccccaggcccctggccccggcgctccccacgtcttctgcctggtc

SEQ ID NO: 5 - protein, 75 aa, Homo sapiens
MVLCYLIRFLQVFVQPANVAVTKMDVSNLAMVMAPNCLRCQSDDPRVIFENTRKEMSFLRVLIQHLDTSFMEGVL

SEQ ID NO: 6 - protein, 159 aa, Homo sapiens
MVLCYLIRFLQVLPSPGVPAAFPSPPRPRPSAPTLAPPPGLRAAGQRRGHQDGCQQPGHGDGAQLLALPVRRPARHLREHPQGDVLPAGAHPAPGHQLHGGCAVAGAPGDRRDVLPPPARPNSALALPAEGPESPGPALEPPPLPQAPGPGAPHVFCLV

(19) United States
(12) Patent Application Publication
(10) Pub. No.: US 2003/0120039 A1
(43) Pub. Date: Jun. 26, 2003

(54) HUMAN PREOPTIC REGULATORY FACTOR-2 AND USES THEREOF

(76) Inventors: Xuan Mei Li, Carmel, IN (US); Paul Robert Rosteck, Indianapolis, IN (US); Eric Wen Su, Carmel, IN (US); He Wang, Carmel, IN (US); Jian-Jun Wei, Edison, NJ (US)

Correspondence Address: ELI LILLY AND COMPANY, PATENT DIVISION, P.O. BOX 6288, INDIANAPOLIS, IN, 46206-6288, US

(21) Appl. No.: 10/129,851

(22) PCT Filed: Nov. 30, 2000
(86) PCT No.: PCT/US00/30169

(30) Foreign Application Priority Data
Dec. 8, 1999 (US) 60169686

(51) Int. Cl. A01K 67/00; C07K 14/705

(52) U.S. Cl. 530/350

(57) ABSTRACT

The present invention relates to novel hPORF-2 polypepiides, including isolated nucleic acids that encode hPORF-2 polypeptides, vectors, host cells, transgenics, chimerics, and hPORF-2-specific antibodies, as well as methods of making and using such.

### BACKGROUND OF THE INVENTION

The present invention relates to compounds and compositions comprising novel human preoptic regulatory factor-2 homologous (hPORF-2) polypeptides, nucleic acids, host cells, transgenics, chimerics, antibodies, compositions, and methods of making and using thereof.

Neuropeptides are central to the regulation of mammalian gender-dependent development and reproduction. The preoptic regulatory factor-2 (PORF-2) is a unique neuropeptide that is expressed in rat brain and testis (Nowak, F. V., Mol. Endocrinol., 4:1205-1210 (1990)). Expression of PORF-2 in specific regions of rat brain including the hypothalamus and hippocampus, is controlled in an age- and gender-dependent manner (Hu, S. B., and Nowak, F. V., Mol. Cell Neurosci, 5, 376-381 (1994); Hu, S. B. , and Nowak, F. V., Endocrine, 3, 421-424 (1995); Nowak, F. V., Torres, G. E., and Hu, S., Neuro. Endocrin., 69:191-201 (1999)). Castration and hypophysectomy results in increased levels of a brain-specific 0.84 kb PORF-2 transcript in the rat preoptic area (POA), but not in the medial basal hypothalamus (MBH) or cerebral cortex (CC). Hypophysectomy also results in a fourfold increase in a 1.1 kb testis-specific transcript. A non-tissue specific 0.6 kb transcript is non-detectable in all tissues examined following hypophysectomy. PORF-2 mRNA is also present in human hypothalamus, testis, adrenal, placenta, and prostate with unique transcripts in each tissue examined. PORF-2 hybridizing transcripts of various sizes, as well as unique, tissue-specific PORF-2 mRNAs, are detected in both rat and human tissues. Interestingly, only a unique single copy PORF-2 gene is present in the rat genome, at least. Thus, the observed transcripts appear to derive from a single DNA locus (Nowak, F. V., Endocrine, 6:57-63 (1997)).

The observed changes in the expression of tissue-specific PORF-2 transcripts in response to hormonal status imply that factors from the pituitary and testis may directly or indirectly influence PORF-2 mRNA transcription, processing, or stability (Nowak, F. V., Endocrine, 6:57-63 (1997). The observation that hypophysectomy resulted in a similar effect suggests this is a direct effect of gonadal factors rather than an indirect one via pituitary hormones.

Tissue-specific processing of mRNAs encoding neuropeptides is well-described (see, Beato, M., Cell, 56:335-344, (1989); Radovick, S., et al., J. Clin. Invest., 88:1649-1655, (1991); Brann, D. W., et al., Neuroendocrin., 54:425-432, (1991); Minn-Worby, C., J. Biol. Chem., 269:15460-15468, (1994); Saht, A., et al., Endocrinology, 130:3331-3336, (1992); Garcia de Yebenes, E., et al., Brain Res., 674: 112-116, (1995)). Generally, alternative transcription, splicing, or processing of mRNAs encoding neuropeptides, in response to intrinsic tissue or cell-type specific signals such as steroids or polypeptide hormones, result in functional consequences by altering translational efficiency, the stability and functional half-life of the mRNAs or transcriptional regulation (see, Girardet, C., et al., Mol. Endocrinol., 10:879-891, (1996); Danoff, A. and Shields, D., J. Biol, Chem., 263:16461-16466, (1988); Kruys, V., et al., Proc. Natl. Acad. Sci. USA, 84:6030-6034, (1987); Garrett, J. E., et al., Mol. Cell Biol., 9:4381-4389 (1989); Shaw, G. and Kamen, R., Cell, 46:659-667, (1986); Lowe, W. L., Jr., et al., Proc. Natl. Acad. Sci. USA, 84:8946-8950, (1987), respectively).

The hypothalamus exercises considerable influence on behavioral, autonomic, visceral, and endocrine functions. For example, hypothalmus function is implicated in the control of water balance, ingestion of food, body temperature and circadian rhythms, sleep, and emotional state. (See generally, Functional Anatomy of the Neuroendocrine Hypothalamus, John Wiley & Sons 1992; Progress in Brain Research Vol 93: The Human Hypothalamus in Health and Disease, Eds. D. F. Swaab et al. Elsevier, 1992). In fact, neuroendocrine cells of the hypothalamus produce more than 20 neuropeptides, including anterior pituitary hormones, opioid peptides, gastrointestinal peptides such as neuropeptide Y and tachykinins, vasoactive peptides such as neurotensin and bradykinin and hypophysiotropic hormones such as thyrotropin-releasing hormone and corticotropin-releasing factor. The central role played by the hypothalamus in regulating mammalian endocrine, autonomic, and behavioral systems is now well recognized and a number of human syndromes have been linked with abnormal function of the hypothalamus. For example, sexual abnormalities, diabetes, psychic disturbances, obesity, somnolence, anorexia and disorders of temperature regulation can all correlate with hypothalamic dysfunction.

Transcriptional modulation of PORF-2 in response to reproductive hormonal status suggests that it may play a important role in hypothalamic pituitary-gonadal interactions. Because of the known links between the hormonal millieu and stress, memory, apetite, metabolism, and sexual behavior, the dependency of PORF-2 transcription upon age, gender, brain region, and gonadal steroid or pituatary polypeptide hormone stimulation there is an interest in and a need for hPORF-2 polypeptides, nucleic acids, host cells, transgenics, chimerics, as well as methods of making and using thereof. Accordingly, we provide novel human homologs of PORF-2.

The present invention provides isolated hPORF-2 nucleic acids, the polypeptides encoded thereby, including specified fragments and variants thereof, hPORF-2 compositions, probes, primers, vectors, host cells, antibodies, transgenics, chimerics, and methods of making and using thereof, as described and enabled herein.

The present invention provides, in one aspect, isolated nucleic acid molecules comprising or complementary to polynucleotides encoding specific hPORF-2 polypeptides, as well as fragments or specified variants thereof.

The novel nucleic acid molecules of the present invention include alternative splicing products of sequences which encode the human homolog of the rat preoptic regulatory factor-2 protein.

The present invention further provides recombinant vectors, comprising 1-40 of said isolated hPORF-2 nucleic acid molecules of the present invention, host cells containing such nucleic acids or recombinant vectors, as well as, methods of making and using such vectors and host cells.

The present invention also provides an isolated hPORF-2 polypeptide, comprising at least one fragment or domain, and specified variants thereof, comprising at least 90-100% of the amino acids in any contiguous portion of at least one of the polypeptides as shown in SEQ ID NOS: 5 or 6.

In another embodiment, the present invention relates to an isolated protein molecule, or functional fragment thereof, wherein said protein molecule comprises at least one of the sequences identified as SEQ ID NOS: 5 or 6. Functional fragments of preference include polypeptides as shown in at least one of SEQ ID NOS: 5 or 6 wherein said polypeptide lacks from 1 to 30 amino acid residues from the amino terminus. More preferable functional fragments are polypeptides as shown in at least one of SEQ ID NOS: 5 or 6 wherein said polypeptide lacks from 1 to 20 amino acid residues from the amino terminus. Most preferred functional fragments are polypeptides as shown in at least one of SEQ ID NOS: 5-6 wherein said polypeptide lacks from 1 to 15 amino acid residues from the amino terminus.

The present invention also provides an isolated hPORF-2 polypeptide as described herein, wherein the polypeptide further comprises at least one specified substitution, insertion or deletion corresponding to portions or residues of at least one of SEQ ID NOS: 5 or 6.

The present invention also provides an isolated hPOPF-2 polypeptide as described herein, wherein the polypeptide has at least one activity such as, but not limited to, promoting neurite outgrowth, inducing neural regeneration, inhibiting neural degeneration, promoting or inhibiting primary or secondary sexual development, and altering behavioral patterns including, but not limited to, sleep and eating disorders. An hPORF-2 polypeptide can therefore be screened for such activities according to known methods.

The present invention also provides a composition comprising an isolated hPORF-2 nucleic acid and/or polypeptide as described herein and a carrier, diluent, or excipient. Optionally, the carrier, diluent, or excipient can be formulated to be pharmaceutically acceptable according to known methods.

Methods for treatment of diseases or disorders using the nucleic acids, polypeptides, or antibodies described are also part of the invention. For instance, a method of treatment or prophylaxis for a nervous disease or disorder can be effected with the polypeptides, nucleic acids, or antibodies described. Similarly, included in the present invention are methods for the prophylaxis or treatment of pathophysiological conditions of the nervous system in which at least one cell type involved in said condition is sensitive or responsive to a polypeptide, nucleic acid, or antibody of the present invention. The present invention includes methods for treatment when the condition involves peripheral nervous system nerve damage, central nervous system nerve damage; neurodegenerative disorders; abnormal primary or secondary sexual development; undesired reproductive disorders including, but not limited to, impotence, infertility, or reduced libido; and undesired behavioral disorders including, but not limited to, sleep or eating disorders. In any of these cases, prophylaxis or treatment comprises administering an effective amount of the polypeptide, nucleic acid, antibody, or pharmaceutically acceptable formulation thereof, to a vertebrate. Preferably, the vertebrate is a mammal. Most preferably, the vertebrate is a human.

The present invention also provides an isolated nucleic acid probe, primer or fragment, as described herein, wherein the nucleic acid comprises a polynucleotide of at least 10 nucleotides, corresponding or complementary to at least 10 nucleotides of at least one of SEQ ID NOS: 1, 2, 3, or 4.

The present invention also provides a recombinant vector comprising an isolated hPORF-2 nucleic acid as described herein.

The present invention also provides a host cell, comprising an isolated hPORF-2 nucleic acid as described herein.

The present invention also provides a method for constructing a recombinant host cell that expresses an hPORF-2 polypeptide, comprising introducing into the host cell an hPORF-2 nucleic acid in replicatable form as described herein to provide the recombinant host cell. The present invention also provides a recombinant host cell provided by a method as described herein.

The present invention also provides a method for expressing at least one hPORF-2 polypeptide in a recombinant host cell comprising culturing a recombinant host cell as described herein under conditions wherein said hPORF-2 polypeptide is expressed in detectable or recoverable amounts.

The present invention provides methods of making and using hPORF-2 nucleic acids, vectors, or host cells for the production of hPORF-2 nucleic acids or hPORF-2 polypeptides, by recombinant, synthetic, or purification techniques which rely on the teachings and guidance presented solely herein or combined with those known in the art.

The present invention also provides an hPORF-2 antibody comprising a polyclonal or monoclonal antibody or fragment thereof that specifically binds at least one epitope specific to at least one of the novel hPORF-2 polypeptides of the present invention.

The present invention also provides a method for producing an antibody or antibody fragment that binds at least one epitope that is specific to at least one of the hPORF-2 polypeptides described herein comprising use of said polypeptide or nucleic acid encoding said polypeptide in recombinant, synthetic, and/or hybridoma methods known in the art to generate said antibody or antibody fragment.

The antibody or antibody fragment of the present invention are useful for therapeutic or diagnostic purposes, or in affinity isolation processes including, but not limited to, affinity chromatography, for the separation of a receptor capable of binding specifically to at least one of the novel polypeptides described herein.

The present invention also provides a method for identifying compounds that bind an hPORF-2 polypeptide, comprising

a) admixing at least one isolated hPORF-2 polypeptide as described herein with a test compound or composition; and

b) detecting at least one binding interaction between the polypeptide and the compound or composition, optionally further comprising detecting a change in biological activity.

The present invention provides isolated, recombinant and/or synthetic nucleic acid molecules comprising at least one polynucleotide encoding at least one hPORF-2 polypeptide, as well as said hPORF-2 polypeptides, and methods of making and using said nucleic acids and polypeptides. An hPORF-2 polypeptide of the present invention is contemplated as comprising at least one fragment, domain, or specified variant of an hPORF-2 polypeptide as shown in SEQ ID NOS: 5 or 6.

The present invention also provides at least one utility by providing isolated nucleic acid comprising polynucleotides of sufficient length and complementarity to an hPORF-2 nucleic acid for use as probes or amplification primers in the detection, quantitation, or isolation of gene sequences or transcripts. For example, isolated nucleic acids of the present invention can be used as probes for detecting deficiencies in the level of mRNA, in screens for detection of mutations in at least one hPORF-2 gene (e.g., substitutions, deletions, or additions), or for monitoring upregulation of expression of said gene, or changes in biological activity as described herein in screening assays of compounds, and/or for detection of any number of allelic variants (polymorphisms or isoforms) of the gene.

The isolated nucleic acids of the present invention can also be used for recombinant expression of hPORF-2 polypeptides. The isolated nucleic acids of the present invention can also be employed for use in sense or antisense suppression of one or more hPORF-2 genes or nucleic acids, in a host cell, or tissue in vivo or in vitro. Attachment of chemical agents which bind, intercalate, cleave and/or crosslink to the isolated nucleic acids of the present invention can also be used to modulate transcription or translation of at least one nucleic acid disclosed herein.

The isolated polypeptides of the present invention can be used as immunogens in the preparation and screening of antibodies directed at hPORF-2 polypeptides.

Citations

All publications or patents cited herein are entirely incorporated herein by reference as they show the state of the art at the time of the present invention to provide description and enablement of the present invention. Publications refer to scientific, patent publication or any other information available in any media format, including all recorded, electronic or printed formats. The following citations are entirely incorporated by reference: Ausubel, et al., eds., Current Protocols in Molecular Biology, John Wiley & Sons, N.Y., N.Y. (1987-1998); Coligan et al., eds., Current Protocols in Protein Science, John Wiley & Sons, Inc., N.Y., N.Y. (1995-1999); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Coligan, et al., eds., Current Protocols in Immunology, John Wiley & Sons, N.Y., N.Y. (1992-1999).

Definitions

The following definitions of terms are intended to correspond to those as well known in the art. The following terms are therefore not limited to the definitions given, but are used according to the state of the art, as demonstrated by cited and/or contemporary publications or patents.

The term “antibody” refers to intact molecules as well as to fragments thereof, such as Fa, F(ab′)₂, and Fv fragments which are capable of binding the eptitopic determinant. Antibodies that bind hPORF-2 polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired.

The terms “complementary” or “complementarity” as used herein refer to the capacity of purine, pyrimidine, synthetic or modified nucleotides to associate by partial or complete complementarity through hydrogen or other bonding to form partial or complete double- or triple-stranded nucleic acid molecules. The following base pairs occur by complete complementarity: (i) guanine (G) and cytosine (C); (ii) adenine (A) and thymine (T); and adenine (A) and uracil (U). “Partial complementarity” refers to association of two or more bases by one or more hydrogen bonds or attraction that is less than the complete complementarity as described above. Partial or complete complementarity can occur between any two nucleotides, including naturally occurring or modified bases, e.g., as listed in 37 CFR § 1.822. All such nucleotides are included in polynucleotides of the invention as described herein. The term “conservative” in reference to an amino acid change or substitution is intended to indicate an amino acid has been replaced with a similar amino acid. Similar amino acids are amino acids that, because of size, charge, polarity and conformation, are more readily substituted without significantly affecting the structure and/or function of the protein. Thus, one skilled in the art generally does not expect a “conservative” amino acid change or substitution to result in any measurable difference in any particular characteristic, property, and/or activity of a polypeptide having a particular conservative amino acid substitution. Specific examples of amino acid changes or substitutions considered to be conservative are known in the art. These examples include, but are not limited to, the non-polar amino acids Glycine, Alanine, Valine, Tsoleucine, and Leucine; the non-polar aromatic amino acids Phenylalanine, Tryptophan, and Tyrosine; the neutral polar amino acids Serine, Threonine, Cysteine, Glutamine, Asparagine, and Methionine; the negatively charged amino acids Lysine, Arginine, and Histidine; the positively charged amino acids Aspartate and Glutamate, represent groups of conservative amino acids. Substitution of any one for another in the same group would generally be considered to be a “conservative” substitution by one skilled in the art (See generally, James D. Watson et al., Molecular Biology of the Gene (1987)).

The term “fusion protein” denotes a hybrid protein molecule not found in nature comprising a translational fusion or enzymatic fusion in which two or more different proteins or fragments thereof are covalently linked on a single polypeptide chain. The term “polypeptide” also includes such fusion proteins.

“Host cell” refers to any eucaryotic, procaryotic, or fusion or other cell or pseudo cell or membrane-containing construct that is suitable for propagating and/or expressing an isolated nucleic acid that is introduced into a host cell by any suitable means known in the art (e.g., but not limited to, transformation or transfection, or the like), or induced to express an endogenous nucleic acid encoding an hPORF-2 polypeptide according to the present invention. The cell can be part of a tissue or organism, isolated in culture or in any other suitable form.

The term “hybridization” as used herein refers to a process in which a partially or completely single-stranded nucleic acid molecule joins with a complementary strand through nucleotide base pairing. Hybridization can occur under conditions of low, moderate or high stringency, with high stringency preferred. The degree of hybridization depends upon, for example, the degree of homology, the stringency conditions, and the length of hybridizing strands as known in the art.

By “isolated” nucleic acid molecule(s) is intended a nucleic acid molecule, DNA, RNA, or both which has been removed from its native or naturally occurring environment. For example, recombinant nucleic acid molecules contained or generated in culture, a vector and/or a host cell are considered isolated for the purposes of the present invention. Further examples of isolated nucleic acid molecules include recombinant nucleic acid molecules maintained in heterologous host cells or purified (partially or substantially) nucleic acid molecules in solution. Isolated RNA molecules include in vivo or in vitro RNA transcripts of the nucleic acid molecules of the present invention. Isolated nucleic acid molecules according to the present invention further include such molecules produced synthetically, purified from or provided in cells containing such nucleic acids, where the nucleic acid exists in other than a naturally occurring form, quantitatively or qualitatively.

“Isolated” used in reference to at least one polypeptide of the invention describes a state of isolation such that the peptide or polypeptide is not in a naturally occurring form and/or has been purified to remove at least some portion of cellular or non-cellular molecules with which the protein is naturally associated. However, “isolated” may include the addition of other functional or structural polypeptides for a specific purpose, where the other peptide may occur naturally associated with at least one polypeptide of the present invention, but for which the resulting compound or composition does not exist naturally.

The term “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally can not be predicted with complete accuracy.

Methods for predicting whether a protein has a SP sequence, as well as the cleavage point for that sequence, are available. The analysis of the amino acid sequence of the proteins described herein indicated the cleavage point is after amino acid 14, preferably between 14-15, as presented in SEQ ID NO: 5, and after amino acid 20, preferably between amino acid 20-21, as presented in SEQ ID NO: 6. As one of ordinary skill would appreciate, however, cleavage sites sometimes vary from organism to organism and cannot be predicted with absolute certainty. Accordingly, the present invention contemplates biologically active hPORF-2 polypeptides having a sequence of 90-100% of the contiguous amino acids sequence shown as shown in either SEQ ID NO: 5 or 6 or which have an N-terminus beginning within 10 residues (i.e., +or −10 residues) of the predicted cleavage point at amino acid 14 or amino acid 20 of SEQ ID NO: 5 or 6, respectively. However, cleavage sites for a secreted protein may be determined experimentally by amino-terminal sequencing of the one or more species of mature proteins found within a purified preparation of the protein.

A “nucleic acid probe,” “oligonucleotide probe,” or “probe” as used herein comprises at least one detectably labeled or unlabeled nucleic acid which hybridizes under specified hybridization conditions with at least one other nucleic acid. This term also refers to a single- or partially double-stranded nucleic acid, oligonucleotide or polynucleotide that will associate with a complementary or partially complementary target nucleic acid to form at least a partially double-stranded nucleic acid molecule. A nucleic acid probe may be an oligonucleotide or a nucleotide polymer. A probe can optionally contain a detectable moiety which may be attached to the end(s) of the probe or be internal to the sequence of the probe, termed a “detectable probe” or “detectable nucleic acid probe.”

A “polynucleotide” comprises at least 10-20 nucleotides of a nucleic acid (RNA, DNA or combination thereof), provided by any means, such as synthetic, recombinant isolation or purification method steps.

A “primer” is a nucleic acid fragment or oligonucleotide which functions as an initiating substrate for enzymatic or synthetic elongation of, for example, a nucleic acid molecule, e.g., using an amplification reaction, such as, but not limited to, a polymerase chain reaction (PCR), as known in the art.

The term “stringency” refers to hybridization conditions for nucleic acids in solution. High stringency conditions disfavor non-homologous base pairing. Low stringency conditions have much less of this effect. Stringency may be altered, for example, by changes in temperature and/or salt concentration, or other conditions, as well known in the art.

A non-limiting example of “high stringency” conditions includes, for example, (a) a temperature of about 42° C., a formamide concentration of about . . . 20%, and a low salt (SSC) concentration, or, alternatively, a temperature of about 65° C., or less, and a low salt (SSPE) concentration; (b) hybridization in 0.5 M NaHPO4, 7% sodium dodecyl sulfate (SDS), 1 mM EDTA at 65° C. (See, e.g., Ausubel, et al., ed., Current Protocols in Molecular Biology, 1987-1998, Wiley Interscience, New York, at §2.10.3). “SSC” comprises a hybridization and wash solution. A stock 20×SSC solution contains 3M sodium chloride, 0.3M sodium citrate, pH 7.0. “SSPE” comprises a hybridization and wash solution. A 1×SSPE solution contains 180 mM NaCl, 9 mM Na2PHO4, 0.9 mM NaH2PO4 and 1 mM EDTA, pH 7.4.

The term “transgene,” as used herein, means a gene which is incorporated into the genome of an animal and is expressed in the animal, resulting in the presence of at least one hPORF-2 polypeptide expressed by the transgenic animal.

The term “variant” when used in herein refers to an amino acid sequence that is altered by one or more amino acids. The variant may have “conservative” changes and/or “non-conservative” changes. Analagous minor variations may also include amino acid deletions or insertions, or both. Guidance in determining which amino acid residues may be substituted, inserted, or deleted without abolishing biological or immunological activity may be found using computer programs well known in the art, for example, DNASTAR software.

The term “vector” as used herein refers to a nucleic acid compound used for introducing exogenous or endogenous nucleic acid into host cells. A vector comprises a nucleotide sequence which may encode one or more polypeptide molecules. Plasmids, cosmids, viruses and bacteriophages, in a natural state or which have undergone recombinant engineering, are non-lirniting examples of commonly used vectors to provide recombinant vectors comprising at least one desired isolated nucleic acid molecule.

Nucleic Acid Molecules

Using the information provided herein, such as the nucleotide sequences encoding at least 90-100% of the contiguous amino acids of at least one of SEQ ID NOS: 5 or 6, specified fragments or variants thereof, or a deposited vector comprising at least one of these sequences, a nucleic acid molecule of the present invention encoding an hPORF-2 polypeptide of the present invention can be obtained using well-known methods.

Nucleic acid molecules of the present invention can be in any form of RNA, such as mRNA, hnRNA, tRNA, or in the form of DNA, including, but not limited to, cDNA and genomic DNA, obtained by cloning or produced synthetically, or any combination thereof. The DNA can be triple-stranded, double-stranded, single-stranded, or any combination thereof. Any portion of at least one strand of the DNA or RNA can be the coding strand, or it can be the non-coding strand, also referred to as the sense or anti-sense strand, respectively.

Isolated nucleic acid molecules of the present invention include nucleic acid molecules comprising an open reading frame (ORF) shown in at least one of SEQ ID NOS: 1, 2, 3, or 4; nucleic acid molecules comprising the coding sequence for an hPORF-2 polypeptide; and nucleic acid molecules which comprise a nucleotide sequence substantially different from those described above but which, due to the degeneracy of the genetic code, still encode at least one hPORF-2 polypeptide as described herein. Because the genetic code is well known in the art, it would be routine for one skilled in the art to generate such degenerate nucleic acid variants that code for specific hPORF-2 polypeptides of the present invention. See, e.g., Ausubel, et al., supra, and such nucleic acid variants are included in the present invention.

In another aspect, the invention provides isolated nucleic acid molecules encoding an hPORF-2 polypeptide having an amino acid sequence as encoded by the cDNA clone contained in the plasmid deposited as designated clone names ______ and ATCC Deposit Nos. ______, respectively, deposited on ______.

In a further embodiment, nucleic acid molecules are provided encoding the mature hPORF-2 polypeptide or an otherwise full-length hPORF-2 polypeptide lacking the N-terminal methionine. The invention also provides an isolated nucleic acid molecule having the nucleotide sequence as shown in any one of SEQ ID NOS: 1, 2, 3, or 4.

Additionally, contemplated isolated nucleic acid molecules are any one of the hPORF-2 polypeptide encoding cDNA sequences contained in at least one of the above-described deposited clones listed herein, as well as any nucleic acid molecule having a sequence complementary thereto. Such isolated molecules, particularly nucleic acid molecules, are useful as probes for gene mapping by in situ hybridization with chromosomes, and for detecting transcription of the hPORF-2 gene in human tissue, for instance, by Northern blot analysis for mRNA detection.

Unless otherwise indicated, all nucleotide sequences identified by sequencing a nucleic acid molecule herein can be or were identified using an automated nucleic acid sequencer, and all amino acid sequences of polypeptides encoded by nucleic acid molecules identified herein can be or were identified by codon correspondence or by translation of a nucleic acid sequence identified using method steps as described herein or as known in the art. It is well known in the art that any nucleic acid sequence identified by this automated approach may contain some errors. Thus, any errors of nucleotide sequence are considered as being reproducibly correctable by resequencing an available or a deposited vector or host cell containing the nucleic acid molecule using well-known methods.

Nucleotide sequences identified by automation are typically at least about 95% to at least about 99.999% identical to the actual nucleotide sequence of the sequenced nucleic acid molecule. The actual sequence can be more precisely identified by other approaches including manual nucleic acid sequencing methods well known in the art. As is also known in the art, a single insertion or deletion in an identified nucleotide sequence compared to the actual sequence will cause a frame shift in translation of the nucleotide sequence such that the identified amino acid sequence encoded by an identified nucleotide sequence will be completely different from the amino acid sequence actually encoded by the sequenced nucleic acid molecule, beginning at the point of such an insertion or deletion.

The present invention is further directed to fragments of the isolated nucleic acid molecules described herein. By a fragment of an isolated nucleic acid molecule is meant a molecule having at least 10 nucleotides of a nucleotide sequence of a deposited cDNA or a nucleotide sequence shown in at least one of SEQ ID NOS: 1, 2, 3, or 4 and is intended to mean fragments at least about 10 nucleotides, and at least about 40 nucleotides in length, which are useful, inter alia as diagnostic probes and primers as described herein. Of course, larger fragments such as at least about 50, 100, 120, 200, 500, 1000, 1500, 2000, 2500, 3000, 3500, and/or 4000 or more nucleotides in length, are also useful according to the present invention as are fragments corresponding to most, if not all, of the nucleotide sequence (or the deposited cDNA) as shown at least one of SEQ ID NOS: 1, 2, 3 or 4. By a fragment at least 10 nucleotides in length, for example, is intended fragments which include 10 or more contiguous nucleotides from the nucleotide sequence of the deposited cDNA or the nucleotide sequence as shown in SEQ ID NOS: 1, 2, 3, 4, or any consensus sequences thereof, as determined by methods known in the art (See e.g., Ausubel, supra, Chapter 7).

Such nucleotide fragments are useful according to the present invention for screening DNA sequences that code for one or more fragments of an hPORF-2 polypeptide as described is herein. Such screening, as a non-limiting example can include the use of so-called “DNA chips” for screening DNA sequences of the present invention of varying lengths, as described, e.g., in U.S. Pat. Nos. 5,631,734, 5,624,711, 5,744,305, 5,770,456, 5,770,722, 5,675,443, 5,695,940, 5,710,000, 5,733,729, which are entirely incorporated herein by reference.

As indicated, nucleic acid molecules of the present invention which comprise a nucleic acid encoding an hPORF-2 polypeptide can include, but are not limited to, those encoding the amino acid sequence of any one of the mature hPORF-2 polypeptides, by itself, as well as the coding sequence for the mature polypeptide having additional sequences. Additional sequences which are contemplated herein include, but are not limited to, the coding sequence of at least one signal leader or fusion peptide. Also contemplated are hPORF-2 polynucleotides encoding the mature polypeptide, with or without additional, non-coding sequences including, but not limited to, introns and non-coding 5′ and 3′ sequences, such as transcribed, non-translated sequences that play a role in transcription, mRNA processing, splicing and polyadenylation signals (for example—ribosome binding and stability of mRNA), or an additional sequence which encodes additional amino acids, including those which provide additional functionalities. Thus, the sequence encoding a polypeptide can be fused to a marker sequence, such as a sequence encoding a peptide that facilitates purification of the fused polypeptide.

Preferred nucleic acid fragments of the present invention also include nucleic acid molecules encoding epitope-bearing portions of an hPORF-2 polypeptide.

In another aspect, the invention provides a polynucleotide (either DNA or RNA) that comprises at least about 20 nt, still more preferably at least about 30 nt, and even more preferably at least about 30-2000 nt of a nucleic acid molecule described herein. These are useful as diagnostic probes and primers as discussed above and in more detail below.

By a portion of a polynucleotide of “at least 10 nt in length,” for example, is intended 10 or more contiguous nucleotides from the nucleotide sequence of the reference polynucleotide (e.g., at least one deposited nucleic acid or at least one nucleotide sequence as shown in at least one of SEQ ID NOS: 1, 2, 3, or 4).

Of course, a polynucleotide which hybridizes only to a poly(A) sequence (such as the 3′ terminal poly(A) of an hPORF-2 cDNA as shown SEQ ID NO: 3) or a complementary stretch of T (or U) resides, would not be included in a probe of the invention, since such a polynucleotide would hybridize to any nucleic acid molecule containing a poly (A) stretch or the complement thereof (e.g., practically any double-stranded cDNA clone).

The present invention also provides subsequences of full-length nucleic acids. Any number of subsequences can be. obtained by reference any one of SEQ ID NOS: 1, 2, 3, 4, or a complementary sequence, and using primers which selectively amplify, under stringent conditions to: at least two sites to the polynucleotides of the present invention, or to two sites within the nucleic acid which flank and comprise a polynucleotide of the present invention, or to a site within a polynucleotide of the present invention and a site within the nucleic acid which comprises it. A variety of methods for obtaining 5′ and/or 3′ ends is well known in the art. See, e.g., RACE (Rapid Amplification of Complementary Ends) as described in M. A. Frohman, PCR Protocols: A Guide to Methods and Applications, M. A. Innis, et al, Eds., Academic Press, Inc., San Diego, Calif., pp. 28-38 (1990); see also, U.S. Pat. No. 5,470,722, and Ausubel, et al., Current Protocols in Molecular Biology, Chapter 15, Eds., John Wiley & Sons, N.Y. (1989-1999). Thus, the present invention provides hPORF-2 polynucleotides having the sequence of the hPORF-2 gene, nuclear transcript, cDNA, or complementary sequences and/or subsequences thereof.

Primer sequences can be obtained by reference to a contiguous subsequence of a polynucleotide of the present invention. Primers are chosen to selectively hybridize, under PCR amplification conditions, to a polynucleotide of the present invention in an amplification mixture comprising a genomic and/or cDNA library from the same species. Generally, the primers are complementary to a subsequence of the amplified nucleic acid. In some embodiments, the primers will be constructed to anneal at their 5′ terminal ends to the codon encoding the carboxy or amino terminal amino acid residue (or the complements thereof) of the polynucleotides of the present invention. The primer length in nucleotides is selected from the group of integers consisting of from at least 15 to 50. Thus, the primers can be at least 15, 18, 20, 25, 30, 40, or 50 nucleotides in length or any range or value therein. A non-annealing sequence at the 5′ end of the primer (a “tail”) can be added, for example, to introduce a cloning site at the terminal ends of the amplified DNA.

The amplification primers may optionally be elongated in the 3′ direction with additional contiguous or complementary nucleotides from the polynucleotide sequences, such as shown in at least one of SEQ ID NOS: 1, 2, 3, or 4 from which they are derived. The number of nucleotides by which the primers can be elongated is selected from the group of integers consisting of from at least 1 to at least 25. Thus, for example, the primers can be elongated with an additional 1, 5, 10, or 15 nucleotides or any range or value therein. Those of skill will recognize that a lengthened primer sequence can be employed to increase specificity of binding (i.e., annealing) to a target sequence, or to add useful sequences, such as links or restriction sites (See e.g., Ausubel, supra, Chapter 15).

The amplification products can be translated using expression systems well known to those of skill in the art and as discussed, infra. The resulting translation products can be confirmed as polypeptides of the present invention by, for example, assaying for the appropriate catalytic activity (e.g., specific activity and/or substrate specificity), or verifying the presence of one or more linear epitopes which are specific to a polypeptide of the present invention. Methods for protein synthesis from PCR derived templates are known in the art (See e.g., Ausubel, supra, Chapters 9, 10, 15; Coligan, Current Protocols in Protein Science, supra, Chapter 5)and available commercially. See, e.g., Amersham Life Sciences, Inc., Catalog '97, p. 354.

The present invention provides isolated nucleic acids that hybridize under selective hybridization conditions to a polynucleotide disclosed herein, e.g., SEQ ID NO: 1. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or CDNA sequences isolated, or otherwise complementary to, a cDNA from a human or mammalian nucleic acid library.

Preferably, the cDNA library comprises at least 80% full-length sequences, preferably at least 85% or 90% full-length sequences, and more preferably at least 95% full-length sequences. The cDNA libraries can be normalized to increase the representation of rare sequences. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this invention will encode an epitope of a polypeptide encoded by the polynucleotides described herein. The polynucleotides of this invention embrace nucleic acid sequences that can be employed for selective hybridization to a polynucleotide encoding a polypeptide of the present invention.

Screening polypeptides for specific binding to antibodies or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278. Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.).

As indicated above, the present invention provides isolated nucleic acids comprising hPORF-2 polynucleotides, wherein the polynucleotides are complementary to the polynucleotides described herein, above. As those of skill in the art will recognize, complementary sequences base pair throughout the entirety of their length with such polynucleotides (i.e., have 100% sequence identity over their entire length). Complementary bases associate through hydrogen bonding in double-stranded nucleic acids. For example, the following base pairs are complementary: guanine and cytosine; adenine and thymine; and adenine and uracil. (See, e.g., Ausubel, supra, Chapter 67; or Sambrook, supra) The isolated nucleic acids of the present invention can be made using (a) standard recombinant methods, (b) synthetic techniques, (c) purification techniques, or combinations thereof, as well known in the art.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention.

Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art (See, e.g., Ausubel, supra, Chapters 1-5; or Sambrook, supra).

The isolated nucleic acid compositions of this invention, such as RNA, cDNA, genomic DNA, or a hybrid thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a CDNA or genomic DNA library. While isolation of RNA, and construction of cDNA and genomic libraries is well known to those of ordinary skill in the art (See, e.g., Ausubel, supra, Chapters 1-7; or Sambrook, supra).

Among other means known by those skilled in the art, the human hPORF-2 gene may be obtained by isolating it from genomic sources, using cDNAs isolated from mRNA templates, by chemical synthesis, or some combination of the preceding methods.

A genomic or cDNA library can be screened using a probe based upon the sequence of a polynucleotide of the present invention, such as those disclosed herein. Probes may be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. Temperature, ionic strength, pH and the presence of a partially denaturing solvent such as formamide can control the degree of stringency. Changing the polarity of the reactant solution through, for example, manipulation of the concentration of formamide within the range of 0% to 50% conveniently varies the stringency of hybridization. The degree of complementarity (sequence identity) required for detectable binding will vary in accordance with the stringency of the hybridization medium and/or wash medium. The degree of complementarity will optimally be 100%; however, it should be understood that minor sequence variations in the probes and primers may be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; 5,142,033 to Innis; 5,122,464 to Wilson, et al.; 5,091,310 to Innis; 5,066,584 to Gyllensten, et al; 4,889,818 to Gelfand, et al; 4,994,370 to Silver, et al; 4,766,067 to Biswas; 4,656,134 to Ringold) and RNA mediated amplification which uses anti-sense RNA to the target sequence as a template for double-stranded DNA synthesis (U.S. Pat. No. 5,130,238 to Malek, et al, with the tradename NASBA), the entire contents of which are herein incorporated by reference. (See, e.g., Ausubel, supra, Chapter 15; or Sambrook, supra) For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, Sambrook, and Ausubel (e.g., Chapter 15) supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products.

The isolated nucleic acids of the present invention can also be prepared by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Letts. 22:1859-1862 (1981); the solid phase phosphoramidite triester method described by Beaucage and Caruthers, Tetra. Letts. 22(20): 1859-1862 (1981), e.g., using an automated synthesizer, e.g., as described in Needham-VanDevanter, et al., Nucleic Acids Res. 12:6159-6168 (1984); and the solid support method of U.S. Pat. No. 4,458,066. Chemical synthesis generally produces a single-stranded oligonucleotide, which may be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences may be obtained by the ligation of shorter sequences.

The present invention further provides recombinant expression cassettes comprising a nucleic acid of the present invention. A nucleic acid sequence of the present invention, for example a cDNA or a genomic sequence encoding a full-length polypeptide of the present invention, can be used to construct a recombinant expression cassette which can be introduced into at least one desired host cell. A recombinant expression cassette will typically comprise a polynucleotide of the present invention operably linked to transcriptional initiation regulatory sequences that will direct the transcription of the polynucleotide in the intended host cell.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter hPORF-2 content and/or composition in a desired tissue.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo or in vitro by mutation, deletion and/or substitution.

A polynucleotide of the present invention can be expressed in either sense or anti-sense orientation as desired. It will be appreciated that control of gene expression in either sense or anti-sense orientation can have a direct impact on the observable characteristics.

Another method of suppression is sense suppression. Introduction of nucleic acid configured in the sense orientation has been shown to be an effective means by which to block the transcription of target genes.

A variety of cross-linking agents, alkylating agents and radical generating species as pendant groups on polynucleotides of the present invention can be used to bind, label, detect and/or cleave nucleic acids. Knorre, et al., Biochimie 67:785-789 (1985); Vlassov, et al., Nucleic Acids Res. 14:4065-4076 (1986); Iverson and Dervan, J. Am. Chem. Soc. 109:1241-1243 (1987); Meyer, et al., J. Am. Chem. Soc. 111:8517-8519 (1989); Lee, et al., Biochemistry 27:3197-3203 (1988); Home, et al., J. Am. Chem. Soc. 112:2435-2437 (1990); Webb and Matteucci, J. Am. Chem. Soc. 108:2764-2765 (1986); Nucleic Acids Res. 14:7661-7674 (1986); Feteritz, et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and 5,681941, each entirely incorporated herein by reference.

The present invention also relates to vectors that include isolated nucleic acid molecules of the present invention, host cells that are genetically engineered with the recombinant vectors, and the production of hPORF-2 polypeptides or fragments thereof by recombinant techniques, as is well known in the art. See, e.g., Sambrook, et al., supra; Ausubel, supra, Chapters 1-9, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, or any other suitable promoter. The skilled artisan will know other suitable promoters. The expression constructs will further contain sites for transcription initiation, termination and, in the transcribed region, a ribosome-binding site for translation. The coding portion of the mature transcripts expressed by the constructs will preferably include a translation initiating codon at the beginning and a termination codon (e.g., UAA, UGA or UAG) appropriately positioned at the end of the mRNA to be translated, with UAA and UAG preferred for mammalian or eukaryotic cell expression.

Expression vectors will preferably include at least one selectable marker. Such markers include, e.g., dihydrofolate reductase, ampicillin (G418), hygromycin or neomycin resistance for eukaryotic cell culture, and tetracycline or ampicillin resistance genes for culturing in E. coli and other bacteria or prokaryotics. Representative examples of appropriate hosts include, but are not limited to, bacterial cells such as E. coli, Streptomyces and Salmonella typhimurium cells; fungal cells such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art. Vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Preferred eucaryotic vectors include pWLNEO, pSV2CAT, pOG44, pXTl and pSG available from Stratagene; and pSVK3, pBPV, pMSG and pSVL available from Pharmacia. Other suitable vectors will be readily apparent to the skilled artisan. See, e.g., Ausubel, supra, Chapter 1; Coligan, Current Protocols in Protein Science, supra, Chapter 5.

Introduction of a vector construct into a host cell can be effected by calcium phosphate transfection, DEAE-dextran mediated transfection, cationic lipid-mediated transfection, electroporation, transduction, infection or other methods. Such methods are described in many standard laboratory manuals, such as Sambrook, supra, Chapters 1-4 and 16-18; Ausubel, supra, Chapters 1, 9, 13, 15, 16.

Polypeptide(s) of the present invention can be expressed in a modified form, such as a fusion protein, and can include not only secretion signals, but also additional heterologous functional regions. For instance, a region of additional amino acids, particularly charged amino acids, can be added to the N-terminus of a polypeptide to improve stability and persistence in the host cell, during purification, or during subsequent handling and storage. Also, peptide moieties can be added to a polypeptide to facilitate purification. Such regions can be removed prior to final preparation of a polypeptide. Such methods are described in many standard laboratory manuals, such as Sambrook, supra, Chapters 17 and 18; Ausubel, supra, Chapters 16, 17 and 18.

Using nucleic acids of the present invention, one may express a protein of the present invention in a recombinantly engineered cell, such as bacteria, yeast, insect, or mammalian cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible) followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present invention. To obtain high level expression of a cloned gene, it is desirable to construct expression vectors which contain, at the minimum, a strong promoter to direct transcription, a ribosome binding site for translational initiation, and a transcription/translation terminator. One of skill would recognize that modifications can be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Alternatively, nucleic acids of the present invention can be expressed in a host cell by turning on (by manipulation) in a host cell that contains endogenous DNA encoding a polypeptide of the present invention. Such methods are well known in the art, e.g., as described in U.S. Pat. Nos. 5,580,734, 5,641,670, 5,733,746, and 5,733,761, entirely incorporated herein by reference.

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., Nature 198:1056 (1977)), the tryptophan (trp) promoter system (Goeddel, et al., Nucleic Acids Res. 8:4057 (1980)) and the lambda derived P L promoter and N-gene ribosome binding site (Shimatake, et al., Nature 292:128 (1981)). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transformed with the plasmid vector DNA. Expression systems for expressing a protein of the present invention are available using Bacillus sp. and Salmonella (Palva, et al., Gene 22:229-235 (1983); Mosbach, et al., Nature 302:543-545 (1983)). See, e.g., Ausubel, supra, Chapters 1-3, 16(Sec.1); and Coligan, supra, Current Protocols in Protein Science, Units 5.1, 6.1-6.7.

A variety of eukaryotic expression systems such as yeast, insect cell lines, plant and mammalian cells, are known to those of skill in the art. As explained briefly below, a nucleic acid of the present invention can be expressed in these eukaryotic systems.

Synthesis of heterologous proteins in yeast is well known. F. Sherman, et al., Methods in Yeast Genetics, Cold Spring Harbor Laboratory (1982) is a well-recognized work describing the various methods available to produce the protein in yeast. Two widely utilized yeast for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant origin. Illustrative of cell cultures useful for the production of the peptides are mammalian cells. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., Immunol. Rev. 89:49 (1986)), and processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7th edition, 1992).

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See Schneider, J. Embryol. Exp. Morphol. 27:353-365 (1987).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VPl intron from SV40 (Sprague, et al., J. Virol. 45:773-781 (1983)). Additionally, gene sequences to control replication in the host cell may be incorporated into the vector such as those found in bovine papilloma virus type-vectors. M. Saveria-Campo, Bovine Papilloma Virus DNA, a Eukaryotic Cloning Vector in DNA Cloning Vol. II, a Practical Approach, D. M. Glover, Ed., IRL Press, Arlington, Va., pp. 213-238 (1985).

An hPORF-2 polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eucaryotic host, including, for example, bacterial, yeast, higher plant, insect and mammalian cells. Depending upon the host employed in a recombinant production procedure, the polypeptides of the present invention can be glycosylated or can be non-glycosylated. In addition, polypeptides of the invention can also include an initial modified methionine residue, in some cases as a result of host-mediated processes. Such methods are described in many standard laboratory manuals, such as Sambrook, supra, Chapters 17.37-17.42; Ausubel, supra, Chapters 10, 12, 13, 16, 18 and 20.

The invention further provides isolated hPORF-2 polypeptides having fragments or specified variants of the amino acid sequence encoded by the deposited cDNAs, or the amino acid sequence in SEQ ID NOS: 5 or 6.

The isolated proteins of the present invention comprise a polypeptide encoded by any one of the polynucleotides of the present invention as discussed more fully, supra, or polypeptides which are specified fragments or variants thereof.

Exemplary polypeptide sequences are provided in SEQ ID NOS: 5 and 6. The proteins of the present invention or variants thereof can comprise any number of contiguous amino acid residues from a polypeptide of the present invention, wherein that number is selected from the group of integers consisting of from 90-100% of the number of contiguous residues in a full-length hPORF-2 polypeptide as shown in at least one of SEQ ID NOS: 5 or 6. Optionally, this subsequence of contiguous amino acids is at least 50, 60, 70, 80, or 90 amino acids in length. Further, the number of such subsequences can be any integer selected from the group consisting of from 1 to 20, such as 2, 3, 4, or 5.

As those of skill will appreciate, the present invention includes biologically active polypeptides of the present invention (i.e., enzymes). Biologically active polypeptides have a specific activity at least 20%, 30%, or 40%, and preferably at least 50%, 60%, or 70%, and most preferably at least 80%, 90%, or 95%-1000% of that of the native (non-synthetic), endogenous polypeptide. Further, the substrate specificity (e.g., k_{cat}/Kₘ) is optionally substantially similar to the native (non-synthetic), endogenous polypeptide. Typically, the Kₘ will be at least 30%, 40%, or 50%, that of the native (non-synthetic), endogenous polypeptide; and more preferably at least 60%, 70%, 80%, or 90%-1000%. Methods of assaying and quantifying measures of enzymatic activity and substrate specificity, are well known to those of skill in the art.

Functional fragments of the proteins disclosed herein may also be identified as having activity. One means of identifying functionally active fragments includes cloning nucleic acid sequences (prepared as described elsewhere herein) which encode fragments of an hPORF-2 into a suitable expression vector, and transforming or transfecting a suitable host cell with the constructs. The culture medium from host cells expressing such fragments can be assayed for at least one of the herein described activities associated with hPORF-2. Activity arising from a known volume of culture medium recovered from a population of cells expressing at least one hPORF-2 polypeptide fragment is normalized with respect to the same activity measured in the same volume of culture medium recovered from a similarly numerous population of control cells known to not express an hPORF-2 polypeptide or fragment thereof. That level of activity is compared to that obtained from the same volume of culture medium recovered from a similarly numerous population of cells known to express a full-length or mature form of at least one of the hPORF-2 polypeptides as shown in SEQ ID NOS: 5 or 6. Fragments of hPORF-2 that retain activity to about 30% or greater of the positive control expressed polypeptide are regarded as biologically functional.

A biologically active hPORF-2 polypeptide of the present invention can include one or more amino acid substitutions, deletions or additions, either from natural mutations or human manipulation, as specified herein.

Of course, the number of amino acid substitutions a skilled artisan would make depends on many factors, including those described above. Generally speaking, the number of amino acid substitutions, insertions or deletions for any given hPORF-2 polypeptide will not be more than 40, 30, 20, 10, 5, or 3, such as 1-30 or any range or value therein, as specified herein.

Amino acids in an hPORF-2 polypeptide of the present invention that are essential for function can be identified by methods known in the art, such as site-directed mutagenesis or alanine-scanning mutagenesis (Cunningham and Wells. Science 244:1081-1085 (1989)). The latter procedure introduces single alanine mutations at every residue in the molecule. The resulting mutant molecules are then tested for biological activity. Sites that are critical for ligand-protein binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)). Non-limiting mutants that can enhance or maintain at least one of the previously mentioned activities include, but are not limited to, any of the above polypeptides, further comprising at least one mutation corresponding to at least one substitution, insertion, or deletion of at least one amino acid as compared to the sequence as shown in SEQ ID NO: 5 or 6.

In another aspect, the invention provides a peptide or polypeptide comprising an epitope-bearing portion of a polypeptide of the invention according to methods well known in the art. See, e.g., Colligan, et al,. ed., Current Protocols in Immunology, Greene Publishing, N.Y. (1993-1998), Ausubel, supra, each entirely incorporated herein by reference.

The epitope of this polypeptide portion is an immunogenic or antigenic epitope of a polypeptide described herein. An “immunogenic epitope” can be defined as a part of a polypeptide that elicits an antibody response when the whole polypeptide is the immunogen. On the other hand, a region of a polypeptide molecule to which an antibody can bind is defined as an “antigenic epitope.” The number of immunogenic epitopes of a polypeptide generally is less than the number of antigenic epitopes. See, for instance, Geysen, et al., Proc. Natl. Acad. Sci. USA 81:3998-4002 (1983).

Generally, the polypeptides of the present invention will, when presented as an immunogen, elicit production of an antibody specifically reactive to a polypeptide of the present invention encoded by a polynucleotide of the present invention as described, supra. Exemplary polypeptides include those which are full-length, such as those disclosed herein. Further, the proteins of the present invention will not bind to antisera raised against a polypeptide of the present invention which has been fully immunosorbed with the same polypeptide. Immunoassays for determining binding are well known to those of skill in the art. A preferred immunoassay is a competitive immunoassay as discussed, infra. Thus, the proteins of the present invention can be employed as immunogens for constructing antibodies immunoreactive to a protein of the present invention for such exemplary utilities as immunoassays or protein purification techniques.

As to the selection of peptides or polypeptides bearing an antigenic epitope (i.e., that contain at least a portion of a region of a polypeptide molecule to which an antibody can bind), it is well known in the art that relatively short synthetic peptides that mimic part of a polypeptide sequence are routinely capable of eliciting an antiserum that reacts with the partially mimicked polypeptide. See, for instance, J. G. Sutcliffe, et al., “Antibodies that react with preidentified sites on polypeptides,” Science 219:660-666 (1983).

Antigenic epitope-bearing peptides and polypeptides of the invention are useful to raise antibodies, including monoclonal antibodies, or screen antibodies, including fragments or single chain antibodies, that bind specifically to a polypeptide of the invention. See, for instance, Wilson, et al., Cell 37:767-778 (1984) at 777. Antigenic epitope-bearing peptides and polypeptides of the invention preferably contain a sequence of at least five, more preferably at least nine, and most preferably between at least about 15 to about 30 amino acids contained within the amino acid sequence of a polypeptide of the invention.

The epitope-bearing peptides and polypeptides of the invention can be produced by any conventional means. R. A. Houghten, “General method for the rapid solid-phase synthesis of large numbers of peptides: specificity of antigen-antibody interaction at the level of individual amino acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten, et al. (1986).

As one of skill in the art will appreciate, hPORF-2 polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker, et al., Nature 331:84-86 (1988)). Fusion proteins that have a disulfide-linked dimeric structure due to the IgG part can also be more efficient in binding and neutralizing other molecules than the monomeric hPORF-2 polypeptide or polypeptide fragment alone (Fountoulakis, et al., J. Biochem. 270:3958-3964 (1995)).

The polypeptides of this invention and fragments thereof may be used in the production of antibodies. The term “antibody” as used herein describes antibodies, fragments of antibodies (such as, but not limited, to Fab, Fab′, Fab2′, and Fv fragments), and modified versions thereof, as well known in the art (e.g., chimeric, humanized, recombinant, veneered, resurfaced or CDR-grafted) such antibodies are capable of binding antigens of a similar nature as the parent antibody molecule from which they are derived. The instant invention also encompasses single chain polypeptide binding molecules.

The production of antibodies, both monoclonal and polyclonal, in animals is well known in the art. See, e.g., Colligan, supra, entirely incorporated herein by reference.

Single chain antibodies and libraries thereof are yet another variety of genetically engineered antibody technology that is well known in the art. (See, e.g., R. E. Bird, et al., Science 242:423-426 (1988); PCT Publication Nos. WO 88/01649, WO 90/14430, and WO 91/10737. Single chain antibody technology involves covalently joining the binding regions of heavy and light chains to generate a single polypeptide chain. The binding specificity of the intact antibody molecule is thereby reproduced on a single polypeptide chain.

Antibodies included in this invention are useful in diagnostics, therapeutics or in diagnostic/therapeutic combinations.

The polypeptides of this invention or suitable fragments thereof can be used to generate polyclonal or monoclonal antibodies, and various inter-species hybrids, or humanized antibodies, or antibody fragments, or single-chain antibodies. The techniques for producing antibodies are well known to skilled artisans. (See, e.g., Colligan supra; Monoclonal Antibodies: Principles & Applications, Ed. J. R. Birch & E. S. Lennox, Wiley-Liss (1995).

A polypeptide used as an immunogen may be modified or administered in an adjuvant, by subcutaneous or intraperitoneal injection into, for example, a mouse or a rabbit. For the production of monoclonal antibodies, spleen cells from immunized animals are removed, fused with myeloma or other suitable known cells, and allowed to become monoclonal antibody producing hybridoma cells in the manner known to the skilled artisan. Hybridomas that secrete a desired antibody molecule can be screened by a variety of well known methods, for example ELISA assay, Western blot analysis, or radioimmunoassay (Lutz, et al. Exp. Cell Res. 175:109-124 (1988); Monoclonal Antibodies: Principles & Applications, Ed. J. R. Birch & E. S. Lennox, Wiley-Liss (1995); Colligan, supra).

For some applications labeled antibodies are desirable. Procedures for labeling antibody molecules are widely known, including for example, the use of radioisotopes, affinity labels, such as biotin or avidin, enzymatic labels, for example horseradish peroxidase, and fluorescent labels, such as FITC or rhodamine (See, e.g., Colligan, supra).

Labeled antibodies are useful for a variety of diagnostic applications. In one embodiment the present invention relates to the use of labeled antibodies to detect the presence of an hPORF-2 polypeptide. Alternatively, the antibodies could be used in a screen to identify potential modulators of an hPORF-2 polypeptide. For example, in a competitive displacement assay, the antibody or compound to be tested is labeled by any suitable method. Competitive displacement of an antibody from an antibody-antigen complex by a test compound such that a test compound-antigen complex is formed provides a method for identifying compounds that bind hPORF-2 polypeptides.

Determination of the ability of compounds to stimulate or inhibit the activity of the hPORF-2 molecule is essential to identifying and developing such compounds for therapeutic applications. Such compounds may be useful as therapeutics, e.g., for treating or preventing the development of diseases or conditions caused by, or contributed to by abnormal hPORF-2 activity, or which can benefit from modulation of an hPORF-2 activity or protein expression level. Thus, the need for bioactivity assay systems that can determine whether a compound affects the function of the hPORF-2 molecule is clear. Accordingly, the present invention provides screening methods for identifying compounds that bind the hPORF-2 protein and/or modify its activity. A non-limiting example of a method for identifying compounds that bind the hPORF-2 protein and/or modifies PORF-2 activity comprises the steps of admixing a substantially purified preparation of an hPORF-2 protein with a test compound, and monitoring by any suitable means a binding interaction between said protein and said compound.

Another example of a method for identifying compounds that bind the hPORF-2 protein or modify its activity comprises the steps of:

a) transfecting a mammalian host cell with an expression vector comprising DNA encoding an hPORF-2 protein;

b) culturing said host cell under conditions such that the hPORF-2 protein is expressed,

c) exposing said host cell so transfected to a test compound, and

d) measuring a change in a physiological condition known to be influenced by the activity of the hPORF-2 protein relative to a control in which the transfected host cell was not exposed to a test compound.

The skilled artisan will recognize that any information regarding the binding potential, inhibitory activity, or selectivity of a particular compound is useful toward the development of pharmaceutical products. For example, a compound with an IC₅₀ which is less than 10 nM is generally considered an excellent candidate for drug therapy. However, a compound which has a lower affinity, but is selective for a particular target, may be an even better candidate.

Another embodiment of the present invention provides transgenic non-human mammals carrying a recombinant human hPORF-2 gene construct in its somatic and germ cells. The recombinant gene construct may be composed of regulatory DNA sequences that belong to the native hPORF-2 gene or those which are derived from an alternative source. These regulatory sequences are functionally linked to the human hPORF-2 coding region, resulting in the constitutive and/or regulatable expression of human hPORF-2 in the body of the transgenic non-human mammal. The most important of such regulatory sequences is the promoter. Promoters are defined in this context as any and all DNA elements necessary for the functional expression of a gene. Promoters drive the expression of structural genes and may be modulated by inducers and repressors. Numerous promoters have been described in the literature and are easily within the grasp of the ordinarily skilled artisan. Viral promoters, such as the SV40 early promoter, are consistent with the invention though mammalian promoters are preferred. The promoter is chosen such that the level of expression is sufficient to promote physiological consequences in the transgenic non-human mammal, or ancestor of said mammal. Preferably, the genome of the transgenic mammal contains at least 30 copies of a transgene. More preferably, the genome of the transgenic mammal contains at least 50 copies, and may contain 100-200 or more copies of the transgene. Generally, said nucleic acid is introduced into said mammal at an embryonic stage, preferably the 1-1000 cell or oocyte stage, and, most preferably not later than about the 64-cell stage. Most preferably the transgenic mammal is homozygous for the transgene.

The techniques described in Leder, U.S. Pat. No. 4,736,866 (hereby entirely incorporated by reference) for producing transgenic non-human mammals may be used for the production of a transgenic non-human mammal of the present invention. The various techniques described in U.S. Pat. Nos. 5,454,807, 5,073,490, 5,347,075 and 4,736,866, the entire contents of which are hereby incorporated by reference, may also be used. Such methods are also described in Methods in Molecular Biology, Vol. 18, 1993, Transgenesis Techniques, Principles and Protocols, (Murphy, D., and Carter, D. A.) as well as in U.S. Pat. Nos. 5,174,986, 5,175,383, 5,175,384, and 5,175,385, all of which are herein incorporated by reference.

Also intended to be within the scope of the present invention are chimeric non-human mammals in which fewer than all of the somatic and germ cells contain a DNA construct comprising a nucleic acid encoding a hPORF-2 polypeptide of the present invention. Contemplated chimeric non-human mammals include animals produced when fewer than all of the cells of the morula are transfected in the process of producing the transgenic animal.

Transgenic and chimeric non-human mammals having human cells or tissue engrafted therein are also encompassed by the present invention. Methods for providing chimeric non-human mammals are provided, e.g., in U.S. Ser. Nos. 07/508,225, 07/518,748, 07/529,217, 07/562,746, 07/596,518, 07/574,748, 07/575,962, 07/207,273, 07/241,590 and 07/137,173, which are entirely incorporated herein by reference, for their description of how to engraft human cells or tissue into non-human mammals.

Alternatively, genetic constructs comprising at least one of the hPORF-2 nucleic acid sequences as defined herein may be used to create transgenic “knockouts” of the hPORF-2 gene. Accordingly, the present invention also provides a transgenic animal which has been engineered by homologous recombination to be deficient in the expression of the endogenous hPORF-2 gene. Further, the invention provides a method of producing an heterozygous or homozygous transgenic animal deficient in or lacking functional PORF-2 proteins, respectfully, said method comprising:

a) obtaining a DNA construct comprising a disrupted hPORF-2 gene, wherein said disruption is by the insertion of an heterologous marker sequence;

b) introducing said DNA construct into an ES cell of said animal such that the endogenous PORF-2 gene is disrupted by homologous recombination;

c) selecting ES cells comprising said disrupted allele;

d) incorporating the ES cells of step c) into a mouse embryo;

e) transferring said embryo into a pseudopregnant animal of the said species;

f) developing said embryo into a viable offspring;

g) screening offspring to identify heterozygous animal comprising said disrupted PORF-2 gene; and

h) if desired, breeding said heterozygous animal to produce homozygous transgenic animals of said species, wherein the said homozygous animal does not express functional PORF-2 proteins.

Transgenic and chimeric non-human mammals of the present invention may be used for analyzing the consequences of over-expression of at least one hPORF-2 polypeptide in vivo. Such animals are also useful for testing the effectiveness of therapeutic and/or diagnostic agents, either associated or unassociated with delivery vectors or vehicles, which preferentially bind to an hPORF-2 polypeptide of the present invention or act to indirectly modulate hPORF-2 activity.

hPORF-2 transgenic non-human mammals are useful as an animal models in both basic research and drug development endeavors. Transgenic animals carrying at least one hPORF-2 polypeptide or nucleic acid can be used to test compounds or other treatment modalities which may prevent, suppress, or cure a pathology or disease associated with at least one of the above mentioned hPORF-2 activities. Such transgenic animals can also serve as a model for the testing of diagnostic methods for those same diseases. Furthermore, tissues derived from hPORF-2 transgenic non-human mammals are useful as a source of cells for cell culture in efforts to develop in vitro bioassays to identify compounds that modulate hPORF-2 activity or hPORF-2 dependent signaling. Accordingly, another aspect of the present invention contemplates a method of identifying compounds efficacious in the treatment of at least one previously described disease or pathology associated with aberrant pre-optic regulatory factor-2 activity. A non-limiting example of such a method comprises:

a) generating an hPORF-2 transgenic non-human animal which is, as compared to a wild-type animal, pathologically distinct in some detectable or measurable manner from wild-type version of said non-human mammal;

b) exposing said transgenic animal to a compound, and;

c) determining the progression of the pathology in the treated transgenic animal, wherein an arrest, delay, or reversal in disease progression in transgenic animal treated with said compound as compared to the progression of the pathology in an untreated control animals is indicative that the compound is useful for the treatment of said pathology

Another embodiment of the present invention provides a method of identifying compounds capable of inhibiting hPORF-2 activity in vivo and/or in vitro wherein said method comprises:

a) administering an experimental compound to an hPORF-2 transgenic non-human animal, or tissues derived therefrom, exhibiting one or more physiological or pathological conditions attributable to the overexpression of an hPORF-2 transgene; and

b) observing or assaying said animal and/or animal tissues to detect changes in said physiological or pathological condition or conditions.

Another embodiment of the invention provides a method for identifying compounds capable of overcoming deficiencies in hPORF-2 activity in vivo or in vitro wherein said method comprises:

a) administering an experimental compound to an hPORF-2 transgenic non-human animal, or tissues derived therefrom, exhibiting one or more physiological or pathological conditions attributable to the disruption of the endogenous PORP-2 gene; and

b) observing or assaying said animal and/or animal tissues to detect changes in said physiological or pathological condition or conditions.

Various means for determining a compound's ability to modulate human hPORF-2 in the body of the transgenic animal are consistent with the invention. observing the reversal of a pathological condition in the transgenic animal after administering a compound is one such means. Another more preferred means is to assay for markers of hPORF-2 activity in the blood of a transgenic animal before and after administering an experimental compound to the animal. The level of skill of an artisan in the relevant arts readily provides the practitioner with numerous methods for assaying physiological changes related to therapeutic modulation of hPORF-2 activity.

In all previously described in vitro and in vivo assays, the experimental compound may be administered when applicable, either superficially, orally, parenterally (e.g. by intravenous infusion or injection) or a combination of injection and infusion (iv), intramuscularly (im), or subcutaneously (sc). A preferred route of compound administration to an animal is iv, while oral administration is most preferred.

By way of illustration, the following examples are provided to help describe how to make and practice the various embodiments of the invention. Those skilled in the art will recognize that the particular reagents, equipment, and procedures described are merely illustrative and are not intended to limit the present invention in any manner.

### EXAMPLE 1

### Expression and Purification of an hPORF-2 Polypeptide in E. coli

The bacterial expression vector pQE60 is used for bacterial expression in this example. (QIAGEN, Inc., Chatsworth, Calif.). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., and suitable single restriction enzyme cleavage sites. These elements are arranged such that a DNA fragment encoding a polypeptide can be inserted in such a way as to produce that polypeptide with the six His residues (i.e., a “6×His tag”) covalently linked to the carboxyl terminus of that polypeptide. However, a polypeptide coding sequence can optionally be inserted such that translation of the six His codons is prevented and, therefore, a polypeptide is produced with no 6×His tag.

The nucleic acid sequence encoding the desired portion of an hPORF-2 polypeptide lacking the hydrophobic leader sequence is amplified from the deposited cDNA clone using PCR oligonucleotide primers (based on the sequences presented, e.g., as presented in at least one of SEQ ID NOS: 1, 2, 3, or 4), which anneal to the amino terminal encoding DNA sequences of the desired portion of an hPORF-2 polypeptide and to sequences in the deposited construct 3′ to the cDNA coding sequence. Additional nucleotides containing restriction sites to facilitate cloning in the pQE60 vector are added to the 5′ and 3′ sequences, respectively.

For cloning an hPORF-2 polypeptide, the 5′ and 3′ primers have nucleotides corresponding or complementary to a portion of the coding sequence of an hPORF-2, e.g., as presented in at least one of SEQ ID NOS: 1, 2, 3, or 4, according to known method steps. One of ordinary skill in the art would appreciate, of course, that the point in a polypeptide coding sequence where the 5 ′ primer begins can be varied to amplify a desired portion of the complete polypeptide shorter or longer than the mature form.

The amplified hPORF-2 nucleic acid fragments and the vector pQE60 are digested with appropriate restriction enzymes and the digested DNAs are then ligated together. Insertion of the hPORF-2 DNA into the restricted pQE60 vector places an hPORF-2 polypeptide coding region including its associated stop codon downstream from the IPTG-inducible promoter and in-frame with an initiating AUG codon. The associated stop codon prevents translation of the six histidine codons downstream of the insertion point.

The ligation mixture is transformed into competent E. coli cells using standard procedures such as those described in Sambrook, et al., 1989; Ausubel, 1987-1998. E. coli strain M15/rep4, containing multiple copies of the plasmid pREP4, which expresses the lac repressor and confers kanamycin resistance (“Kanrl”), is used in carrying out the illustrative example described herein. This strain, which is only one of many that are suitable for expressing hPORF-2 polypeptide, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH8. The cell debris is removed by centrifugation, and the supernatant containing the hPORF-2 is dialyzed against 50 mM Na-acetate buffer pH6, supplemented with 200 rnM NaCl. Alternatively, a polypeptide can be successfully refolded by dialyzing it against 500 mm NaCl, 20% glycerol, 25 mM Tris/HCI pH7.4, containing protease inhibitors.

If insoluble protein is generated, the protein is made soluble according to known method steps. After renaturation the polypeptide is purified by ion exchange, hydrophobic interaction and size exclusion chromatography. is Alternatively, an affinity chromatography step such as an antibody column is used to obtain pure hPORF-2 polypeptide. The purified polypeptide is stored at 4° C. or frozen at −40° C. to −120° C.

### EXAMPLE 2

### Cloning and Expression of an hPORF-2 Polypeptide in a Baculovirus Expression System

In this illustrative example, the plasmid shuttle vector pA2 GP is used to insert the cloned DNA encoding the mature polypeptide into a baculovirus to express an hPORF-2 polypeptide, using a baculovirus leader and standard methods as described in Summers, et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by the secretory signal peptide (leader) of the baculovirus gp67 polypeptide and convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Other baculovirus vectors are used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow, et al., Virology 170:31-39.

The cDNA sequence encoding the mature hPORF-2 polypeptide in the deposited or other clone, lacking the AUG initiation codon and the naturally associated nucleotide binding site, is amplified using PCR oligonucleotide primers corresponding to the 5′ and 3′ sequences of the gene. Non-limiting examples include 5′ and 3′ primers having nucleotides corresponding or complementary to a portion of the coding sequence of an hPORF-2 polypeptide, e.g., as presented in at least one of SEQ ID NOS: 1, 2, 3, or 4, according to known method steps.

The amplified fragment is isolated from a 1% agarose gel using a commercially available kit (e.g., “Geneclean,” BIO 101 Inc., La Jolla, Calif.). The fragment then is then digested with the appropriate restriction enzyme and again is purified on a 1% agarose gel. This fragment is designated herein “F1”.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria are identified that contain the plasmid with the human hPORP-2 gene using the PCR method, in which one of the primers that is used to amplify the gene and the second primer is from well within the vector so that only those bacterial colonies containing the hPORF-2 gene fragment will show amplification of the DNA. The sequence of the cloned fragment is confirmed by DNA sequencing. This plasmid is designated herein pBac hPORF-2

Five μg of the plasmid pBachPORF-2 is co-transfected with 1.0 μg of a commercially available linearized baculovirus DNA (“BaculoGold™ baculovirus DNA”, Pharmingen, San Diego, Calif.), using the lipofection method described by Felgner, et al., Proc. Natl. Acad. Sci. USA 84:7413-7417 (1987). 1 μg of BaculoGold™ virus DNA and 5 μg of the plasmid pBac hPORF-2 are mixed in a sterile well of a microtiter plate containing 50 μl of serum-free Grace's medium (Life Technologies, Inc., Rockville, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed, according to known methods. An agarose gel with “Blue Gal” (Life Technologies, Inc., Rockville, Md.) is used to allow easy identification and isolation of gal-expressing clones, which produce blue-stained plaques. (A detailed description of a “plaque assay” of this type can also be found in the user's guide for insect cell culture and baculovirology distributed by is Life Technologies, Inc., Rockville, Md., page 9-10). After appropriate incubation, blue stained plaques are picked with a micropipettor tip (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-hPORP-2.

To verify the expression of the hPORF-2 gene, Sf9 cells are grown in Grace's medium supplemented with 10% heat-inactivated FBS. The cells are infected with the recombinant baculovirus V-hPORF-2 at a multiplicity of infection (“MOI”) of about 2. Six hours later the medium is removed and is replaced with SF900 II medium minus methionine and cysteine (available, e.g., from Life Technologies, Inc., Rockville, Md.). If radiolabeled polypeptides are desired, 42 hours later, 5 mCi of 35S-methionine and 5 mCi 35S-cysteine (available from Amersham) are added. The cells are further incubated for 16 hours and then they are harvested by centrifugation. The polypeptides in the supernatant as well as the intracellular polypeptides are analyzed by SDS-PAGE followed by autoradiography (if radiolabeled). Microsequencing of the amino acid sequence of the amino terminus of purified polypeptide can be used to determine the amino terminal sequence of the mature polypeptide and thus the cleavage point and length of the secretory signal peptide.

### EXAMPLE 3

### Cloning and Expression of hPoRF-2 in Mammalian Cells

A typical mammalian expression vector contains at least one promoter element, which mediates the initiation of transcription of mRNA, the polypeptide coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e-g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRESlneo, pRetro-Off, pRetro-On, PLXSN, or PLNCX (Clonetech Labs, Palo Alto, Calif.), PcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded polypeptide. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using-these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp7l8, facilitate the cloning of the gene of interest. The vectors contain in addition the 3′ intron, the polyadenylation and termination signal of the rat preproinsulin gene.

### EXAMPLE 3(a)

### Cloning and ExpreBsion in COS Cells

The expression plasmid, phPORF-2 HA, is made by cloning a CDNA encoding hPORF-2 into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eucaryotic cells; (4) a CMV promoter, a polylinker, an SV40 intron; (5) several codons encoding a hemagglutinin fragment (i.e., an “HA” tag to facilitate purification) or HIS tag (see, e.g, Ausubel, supra) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide described by Wilson, et al., Cell 37:767-778 (1984). The fusion of the HA tag to the target polypeptide allows easy detection and recovery of the recombinant polypeptide with an antibody that recognizes the HA epitope. pcDNAIII contains, in addition, the selectable neomycin marker.

A DNA fragment encoding the hPORF-2 is cloned into the polylinker region of the vector so that recombinant polypeptide expression is directed by the CMV promoter. The plasmid construction strategy is as follows. The hPORF-2 cDNA of the deposited clone is amplified using primers that contain convenient restriction sites, much as described above for construction of vectors for expression of hPORF-2 in E. coli. Non-limiting examples of suitable primers include those based on the coding sequences presented in at least one of SEQ ID NOS: 1, 2, 3, or 4 as they encode hPORF-2 polypeptides as described herein.

The PCR amplified DNA fragment and the vector, pcDNAI/Amp, are digested with suitable restriction enzyme(s) and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the hPORF-2-encoding fragment.

For expression of recombinant hPORF-2, COS cells are transfected with an expression vector, as described above, using DEAE-DEXTRAN, as described, for instance, in Sambrook, et al., Molecular Cloning: a Laboratory Manual, Cold Spring Laboratory Press, Cold Spring Harbor, New York (1989). Cells are incubated under conditions for expression of hPORF-2 by the vector.

Expression of the hPORF-2-HA fusion polypeptide is detected by radiolabeling and immunoprecipitation, using methods described in, for example Harlow, et al., Antibodies: A Laboratory Manual, 2nd Ed., Cold Spring Harbor Laboratory Press, Cold Spring Harbor, New York (1988). To this end, two days after transfection, the cells are labeled by incubation in media containing 35S-cysteine for 8 hours. The cells and the media are collected, and the cells are washed and lysed with detergent-containing RIPA buffer: 150 mM NaCl, 1% NP-40, 0.1% SDS, 0.5% DOC, 50 mM TRIS, pH 7.5, as described by Wilson, et al. cited above. Proteins are precipitated from the cell lysate and from the culture media using an HA-specific monoclonal antibody. The precipitated polypeptides then are analyzed by SDS-PAGE and autoradiography. An expression product of the expected size is seen in the cell lysate, which is not seen in negative controls.

### EXAMPLE 3(b)

### Cloning and Expression in CHO Cells

The vector pC4 is used for the expression of hPORF-2 polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp7l8 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the hPORF-2 in a regulated way in mammalian cells (M. Gossen, and H. Bujard, Proc Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete hPORF-2 polypeptide is amplified using PCR oligonucleotide primers corresponding to the 5′ and 3′ sequences of the gene. Non-limiting examples include 5′ and 3′ primers having nucleotides corresponding or complementary to a portion of the coding sequence of an hPORF-2, e.g., as presented in at least one of SEQ ID NOS: 1, 2, 3, or 4 according to known method steps.

The amplified fragment is digested with suitable endonucleases and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained which grow at a concentration of 100-200 mM. Expression of the desired gene product is analyzed, for instance, by SDS-PAGE and Western blot or by reverse phase HPLC analysis.

### EXAMPLE 4

### Tissue Distribution of hPORF-2 mRNA Expression

Northern blot analysis is carried out to examine hPORF-2 gene expression in human tissues; using methods described by, among others, Sambrook, et al., cited above. A CDNA probe containing the entire nucleotide sequence of an hPORF-2 polypeptide (SEQ ID NOS:L) is labeled with ³²p using the Rediprime™ DNA labeling system (Amersham Life Science), according to the manufacturer's instructions. After labeling, the probe is purified using a CHROMA SPIN-100™ column (Clontech Laboratories, Inc.), according to the manufacturer's protocol number PT1200-1. The purified and labeled probe is used to examine various human tissues for hPORF-2 mRNA.

Multiple Tissue Northern (MTN) blots containing various human tissues (H) or human immune system tissues (IM) are obtained from Clontech and are examined with the labeled probe using ExpressHyb hybridization solution (Clontech) according to manufacturer's protocol number PT1190-1. Following hybridization and washing, the blots are mounted and exposed to film at −70° C. overnight, and films developed according to standard procedures. Expression of hPORF-2 mRNA transcripts may be detected in brain, testis, and other tissues.

### EXAMPLE 5

### PCR Directed Mutagenesis of hPORF-2 polynucleotides to provide DNA encoding specified substitutions, insertions, or deletions of SEQ ID NO: 1.

The polymerase chain reaction (PCR) can be used for the enzymatic amplification and direct sequencing of small quantities of nucleic acids (see, e.g., Ausubel, supra, section 15) to provide specified substitutions, insertions or deletions in DNA encoding an hPORF-2 polypeptide of the present inventions, e.g., SEQ ID NO: 1, 2, 3, 4, or any sequence described herein, as presented herein, to provide an hPORF-2 polypeptide sequence of interest including at least one substitution, insertion, or deletion from that which is shown as SEQ ID NOS: 4 or 5. This technology can be used as a quick and efficient method for introducing any desired sequence change into the DNA of interest.

Unit 8.5 of Ausubel, supra, contains two basic protocols for introducing base changes into specific DNA sequences. Basic Protocol 1, as presented in the first section 8.5 of Ausubel, supra (entirely incorporated herein by reference), describes the incorporation of a restriction site and Basic Protocol 2, as presented below and in the second section of Unit 8.5 of Ausubel, supra, details the generation of specific point mutations (all of the following references in this example are to sections of Ausubel et al., eds., Current Protocols in Molecular Biology, Wiley Interscience, New York (1987-1999)). An alternate protocol describes generating point mutations by sequential PCR steps. Although the general procedure is the same in all three protocols, there are differences in the design of the synthetic oligonucleotide primers and in the subsequent cloning and analyses of the amplified fragments.

The PCR procedure described here can rapidly, efficiently, and/or reproducibly introduce any desired change into a DNA fragment. It is similar to the oligonucleotide-directed mutagenesis method described in UNIT 8.1, but does not require the preparation of a uracil-substituted DNA template.

The main disadvantage of PCR-generated mutagenesis is related to the fidelity of the Taq DNA polymerase. The mutation frequency for Taq DNA polymerase was initially estimated to be as high as 1/5000 per cycle (Saiki et al., 1988). This means that the entire amplified fragment must be sequenced to be sure that there are no Taq-derived mutations. To reduce the amount of sequencing required, it is best to introduce the mutation by amplifying as small a fragment as possible. With rapid and reproducible methods of double-stranded DNA sequencing (UNIT 7.4), the entire amplified fragment can usually be sequenced from a single primer. If the fragment is somewhat longer, it is best to subclone the fragment into an M13-derived vector, so that both forward and reverse primers can be used to sequence the amplified fragment.

If there are no convenient restriction sites flanking the fragment of interest, the utility of this method is somewhat reduced. Many researchers prefer the mutagenesis procedure in UNIT 8.1 to avoid excessive sequencing.

A full discussion of critical parameters for PCR amplification can be found in UNIT 15.1. Anticipated Results

Each of the procedures presented here has a 100% efficiency rate. All or substantially all of the cloned, amplified fragments will contain the mutation corresponding to the synthesized oligonucleotide.

Literature Cited Saiki, R. K., Gelfand, D. H., Stoffel, S., Scharf, S. J., Higuchi, R., Horn, G. T., Mullis, K. B., and Erlich, H. A. 1988. Primer-directed enzymatic amplification of DNA with a thermostable DNA polymerase. Science 239:487-491.

Basic Protocol (2): Introduction of Point Mutations by PCR

In this protocol, synthetic oligonucleotides are designed to incorporate a point mutation at one end of an amplified fragment. Following PCR, the amplified fragments are made blunt-ended by treatment with Klenow fragment. These fragments are then ligated and subcloned into a vector to facilitate sequence analysis. This procedure is summarized in FIG. 8.5.2 of Ausubel, supra.

Materials

DNA sample to be mutagenized, Klenow fragment of E. coli DNA polymerase I (UNIT 3.5 of Ausubel, supra), appropriate restriction endonucleases (Table 8.5.1), additional reagents and equipment for synthesis and purification of oligonucleotides (UNITS 2.11 & 2.12), phosphorylation of oligonucleotides (UNIT 3.10), electrophoresis of DNA on nondenaturing agarose and low gelling/melting agarose gels (UNITS 2.5A & 2.6), restriction endonuclease digestion (UNIT 3.1), ligation of DNA fragments (UNIT 3.16), transformation of E. coli (UNIT 1.8), plasmid DNA miniprep (UNIT 1.6), and DNA sequence analysis (UNIT 7.4).

Template DNA and Oligonucleotide Primers Preparation

Prepare template DNA (see Basic Protocol 1, steps 1 and 2). Synthesize (UNIT 2.11) and purify (UNIT 2.12) the oligonucleotide primers (primers 3 and 4 in FIG. 8.5.2B). The oligonucleotide primers must be homologous to the template DNA for more than 15 bases. No four-base “clamp” sequence is added to these primers. The primer sequences are based on a DNA encoding the hPORF-2 polypeptide sequence of interest including at least one substitution, insertion or deletion from the amino acid sequence as shown in any one of SEQID NOS: 5 or 6. The 5′ end of the oligonucleotides are phosphorylated so that the 5′ end of the oligonucleotide can be used directly in cloning (UNIT 3.10).

DNA Amplification and Preparation of Blunt-end Fragments

Amplify the template DNA (see Basic•Protocol 1, steps 5 and 6). After the final extension step, add 5 U Klenow fragment to the reaction mix and incubate 15 min at 30° C. During PCR, the Taq polymerase adds an extra nontemplated nucleotide to the 3′ end of the fragment. The 3′-5′ exonuclease activity of the Klenow fragment is required to make the ends flush and suitable for blunt-end cloning (UNIT 3.5). Analyze and process the reaction mix (see Basic Protocol 1, steps 7 and 8). Digest half the amplified fragments with the restriction endonucleases for the flanking sequences (UNIT 3.1). Purify digested fragments on a low gelling/melting agarose gel (UNIT 2.6).

Subclone the two amplified fragments into an appropriately digested vector by blunt-end ligation (UNIT 3.16). Transform recombinant plasmid into E. coli (UNIT 1.8). Prepare DNA by plasmid miniprep (UNIT 1.6). Analyze the amplified fragment portion of the plasmid DNA by DNA sequencing to confirm the point mutation (UNIT 7.4). This is critical because the Taq DNA polymerase can introduce additional mutations into the fragment (see Critical Parameters).

Alternate Protocol: Introdution of a Point Mutation by Sequential PCR Steps

In this procedure, the two fragments encompassing the mutation are annealed with each other and extended by mutually primed synthesis; this fragment is then amplified by a second PCR step, thereby avoiding the blunt-end ligation required in Basic Protocol 2. This strategy is outlined in FIG. 8.5.3. For materials, see Basic Protocols 1 and 2 of Ausubel, supra.

Prepare template DNA (see Basic Protocol 1, steps 1 and 2). Synthesize (UNIT 2.11) and purify (UNIT 2.12) the oligonucleotide primers (primers 5 and 6 in FIG. 8.5.3B) to generate an hPORF-2 polypeptide sequence of interest including at least one substitution, insertion, or deletion from at least one of the amino acid sequences as shown in SEQID NOS: 5 or 6. The oligonucleotides must be homologous to the template for 15 to 20 bases and must overlap with one another by at least 10 bases. The 5′ end does not have a “clamp” sequence.

Amplify the template DNA and generate blunt-end fragments (see Basic Protocol 2, steps 4 and 5). Purify the fragments by nondenaturing agarose gel electrophoresis (UNIT 2.5A). Resuspend in TE buffer at 1 ng/μl.

Carry out second PCR amplification. Combine the following in a 500-μl microcentrifuge tube:

10 μl (10 ng) each amplified fragment

1 μl (500 ng) each flanking sequence primer (each 1 μm final)

10 μl 10×amplification buffer

10 μl 2 mM 4dNTP mix

H₂O to 99.5 μl

0.5 μl Taq DNA polymerase (5 U/μl).

overlay with 100 ul mineral oil. Carry out PCR for 20 to 25 cycles, using the conditions for introduction of restriction endonuclease sites by PCR (see Basic Protocol 1, step 6). Analyze and process the reaction mix (see Basic Protocol 1, Ausubel, supra, steps 7 and 8).

Digest the DNA fragment with the appropriate restriction endonuclease for the flanking sites (UNIT 3.1). Purify the digested fragment on a low gelling/melting agarose gel (UNIT 2-6). Subclone into an appropriately digested vector. Transform recombinant plasmid into E. coli (UNIT 1.8). Prepare DNA by plasmid miniprep (UNIT 1.6). Analyze the amplified fragment portion of the plasmid DNA by DNA sequencing (UNIT 7.4) to confirm the point mutation. This is critical because the Taq DNA polymerase can introduce additional mutations into the fragment (see Critical Parameters).

It will be clear that the present invention can be practiced otherwise than as particularly described in the foregoing description and examples. Numerous modifications and variations of the present invention are possible in light of the above teachings and, therefore, are within the scope of the appended claims.

We claim:

1. An isolated nucleic acid comprising at least one hPORF-2 polynucleotide encoding at least 90-100% of the contiguous amino acids of a protein sequence as shown in at least one of SEQ ID NOS: 5 or 6.

2. An isolated nucleic acid of claim 1 wherein the isolated nucleic acid further comprises at least one mutation corresponding to at least one substitution, insertion or deletion of in a polypeptide sequence as shown in at least one of SEQ ID NOS: 5 or 6.

3. An isolated nucleic acid, comprising at least one hPORF-2 polynucleotide comprising or complementary to at least 90-100% of the contiguous nucleotides of at least one of SEQ ID NOS: 1, 2, 3, or 4.

4. A composition, comprising at least one isolated nucleic acid according to any of claims 1-3 and a carrier or diluent.

5. A recombinant vector, comprising at least one nucleic acid according to any of claims 1-3.

6. A host cell comprising at least one recombinant vector according to claim 5.

7. A method for producing at least one hPORF-2 polypeptide, comprising culturing a host cell according to claim 6 under conditions that the at least one hPORF-2 polypeptide is expressed in detectable or recoverable amounts.

8. A transgenic or chimeric non-human animal, comprising at least one isolated nucleic acid according to any of claims 1-3.

9. An isolated polypeptide comprising an hPORF-2 polypeptide comprising at least 90-100% of the contiguous amino acids of at least one amino acid sequence of SEQ ID NOS: 5 or 6.

10. An isolated polypeptide according to claim 9, wherein said polypeptide further comprises at least one mutation corresponding to at least one substitution, insertion, or deletion of at least one amino acid.

11. An isolated polypeptide comprising at least one polypeptide comprising at least 90-100% of the contiguous amino acids of at least one extracellular, intracellular, transmembrane or active domain of at least one of SEQ ID NOS: 5 or 6.

12. A composition, comprising at least one isolated polypeptide according to any of claims 8-11 and a carrier or diluent.

13. An isolated nucleic acid probe, fragment, or primer, comprising an hPORF-2 polynucleotide comprising a sequence corresponding or complementary to at least 10 nucleotides of any one of SEQ ID NOS: 1, 2, 3, or 4.

14. An isolated nucleic acid, comprising a nucleic acid that hybridizes under stringent conditions to a nucleic acid according to claim 13.

15. An antibody or at least one fragment thereof that binds an epitope specific to at least one hPORF-2 polypeptide according to any of claims 8-11.

16. A host cell, expressing at least one antibody or at least one fragment thereof according to claim 15.

17. A method for producing at least one antibody, comprising culturing a host cell according to claim 16.

18. A method for identifying compounds that bind at least one hPORF-2 polypeptide, comprising
(a) admixing at least one isolated hPORF-2 polypeptide with at least one test compound or composition; and
(b) detecting at least one binding interaction between said at least one hPORF-2 polypeptide and the test compound or composition.

19. A method of identifying compounds capable of inhibiting hPORF-2 activity wherein said method comprises:
a) administering an experimental compound to 1) an hPORF-2 transgenic non-human animal exhibiting one or more physiological, pathological, psychological, or behavioral conditions attributable to the overexpression of an hPORF-2 transgene or 2) tissues derived therefrom; and
b) observing or assaying said animal and/or animal tissues to detect changes in said condition or conditions.

20. A method of identifying compounds capable of overcoming deficiencies in hPORF-2 activity wherein said method comprises the steps of:
a) administering an experimental compound to 1) an hPORF-2 transgenic non-human animal exhibiting one or more physiological, pathological, psychological, or behavioral conditions attributable to the disruption of the endogenous PORF-2 gene; or 2) tissues derived therefrom; and
b) observing or assaying said animal or animal tissues to detect changes in said condition or conditions.

21. A method of identifying a compound which can modulate the activity of the hPORF-2 protein wherein said method comprises the steps of:
a) transfecting a mammalian host cell with an expression vector comprising DNA encoding an hPORF-2 protein;
b) culturing said host cell under conditions such that the hPORF-2 protein is expressed;
c) exposing said host cell so transfected to a test compound; and
d) measuring a change in a physiological condition known to be influenced by the activity of the hPORF-2 protein relative to transfected host cells not exposed to said test compound.

22. The use of a polypeptide selected from the group consisting of:
a) a polypeptide as shown in SEQ ID NO: 5;
b) a polypeptide as shown in SEQ ID NO: 6;
c) a polypeptide which is at least 90% identical to a polypeptide as in a) or b);
d) any fragment of a polypeptide as in a), b), or c) for the manufacture of a medicament for the treatment or prevention of a disease in which aberrant levels of endogenous, PORF-2 polypeptide are at least partially responsible for inducing the cellular effects that lead to said disease.

23. The use of an antisense nucleic acid molecule which is complementary to at least 15 contiguous nucleotides of an mRNA molecule encoded by a polynucleotide selected from the group consisting of:
a) a polynucleotide which has at least 90% identity to at least 40 contiguous nucleotides of at least one of SEQ ID NOS: 1, 2, 3, or 4;
b) a polynucleotide which has at least 95% identity to at least 40 contiguous nucleotides of at least one of SEQ ID NOS: 1, 2, 3, or 4; and
c) a polynucleotide as shown in at least one of SEQ ID NOS: 1, 2, 3, or 4;
whereby said compound binds to said mRNA such that translation of said mRNA is inhibited for the manufacture of a medicament for the treatment or prevention of a disease in which aberrant levels of endogenous PORF-2 polypeptide are at least partially responsible for inducing the cellular effects that lead to said disease.

24. claim 22 or 23 wherein said disease is selected from the group consisting of peripheral nervous system nerve damage, central nervous system nerve damage; neurodegeneration; abnormal primary or secondary sexual development, impotence, infertility, reduced libido, narcolepsy, apnea, anorexia nervosa, and bulimia.

25. A pharmaceutical composition containing a polypeptide selected from the group consisting of:
a) a polypeptide as shown in SEQ ID NO: 5;
b) a polypeptide as shown in SEQ ID NO: 6;
c) a polypeptide which is at least 90% identical to a polypeptide as in a) or b); and
d) any fragment of a polypeptide as in a), b), or c) for treating or preventing a disease in which aberrant levels of endogenous PORF-2 polypeptide are at least partially responsible for inducing the cellular effects that lead to said disease.

26. A pharmaceutical composition containing an antisense nucleic acid molecule which is complementary to at least 15 contiguous nucleotides of an mRNA molecule encoded by a polynucleotide selected from the group consisting of:
a) a polynucleotide which has at least 90% identity to at least 40 contiguous nucleotides of at least one of SEQ ID NOS: 1, 2, 3, or 4;
b) a polynucleotide which has at least 95% identity to at least 40 contiguous nucleotides of at least one of SEQ ID NOS: 1, 2, 3, or 4; and
c) a polynucleotide as shown in at least one of SEQ ID NOS: 1, 2, 3, or 4; whereby said compound binds to said mRNA such that translation of said mRNA is inhibited for the manufacture of a medicament for treating or preventing a disease in which aberrant levels of endogenous PORF-2 polypeptide are, at least partially, responsible for inducing the cellular effects that lead to said disease.

27. claim 25 or 26 wherein said disease is selected from the group consisting of peripheral nervous system nerve damage, central nervous system nerve damage; neurodegeneration; abnormal primary or secondary sexual development, impotence, infertility, reduced libido, narcolepsy, apnea, anorexia nervosa, and bulimia.
